(19) United States
(12) Patent Application Publication
(10) Pub. No.: US 2008/0077100 A1
(43) Pub. Date: Mar. 27, 2008

(54) Urinal which can detect body temperature

(76) Inventor: Olympus Jan, Hualien (TW)

Correspondence Address: PRO-TECHTOR INTERNATIONAL SERVICES, 20775 NORADA CT., SARATOGA, CA, 95070, US

(21) Appl. No.: 11/527,345

(22) Filed: Sep. 26, 2006

(51) Int. Cl. A47K 11/00; A61F 5/44

(52) U.S. Cl. 604/349; 4/144.1

(57) ABSTRACT

A urinal which can detect body temperature uses primarily a habit that an animal will excrete periodically, and a principle that the body temperature is equal to urine temperature, to install a sensor and a temperature displayer on a standing or squatting urinal which is commonly used in a daily life, such that when a user excretes in the urinal, and the urine is in touch with the sensor, message of the body temperature can be transmitted to the temperature displayer for displaying, in order to acquire a physiological condition.

### BACKGROUND OF THE INVENTION

a) Field of the Invention

The present invention relates to a urinal which can detect body temperature, and more particularly to a urinal on which is installed with a sensor and a temperature displayer, such that when a human urine is in touch with the sensor, the body temperature can be displayed on the temperature displayer.

b) Description of the Prior Art

Measuring body temperature to acquire a physiological condition of human is a very important and necessary task. However, in consideration of an inconvenience in carrying a clinical thermometer personally and an accuracy of measurement, an ordinary person seldom has a habit of measuring the body temperature. Nevertheless, when a global influenza, the SARS (Severe Acute Respiratory Syndrome), the foot-and-mouth disease or the avian influenza is spreading, the body temperature must be measured at an ordinary institute, a school, a factory, or a public place. Therefore, a convenient and quick body temperature measurement appliance, such as an ear thermometer, has arisen accordingly. However, as the ear thermometer is very expensive, and is not convenient to carry, it is not very popular. Therefore, if there is no epidemic disease, the general population will not take the body temperature usually.

### SUMMARY OF THE INVENTION

The primary object of present invention is to install a sensor and a temperature displayer on a urinal, by using a habit that an animal will excrete periodically and a principle that the body temperature is equal to urine temperature, such that a most convenient method can be used to measure the body temperature by using the habit that a person must excrete the urine every day, in order to acquire a physiological condition of human body at any time.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a schematic view of a standing urinal.

FIG. 2 shows a schematic view of a squatting urinal.

### DETAILED DESCRIPTION OF THE PREFERRED EMBODIMENTS

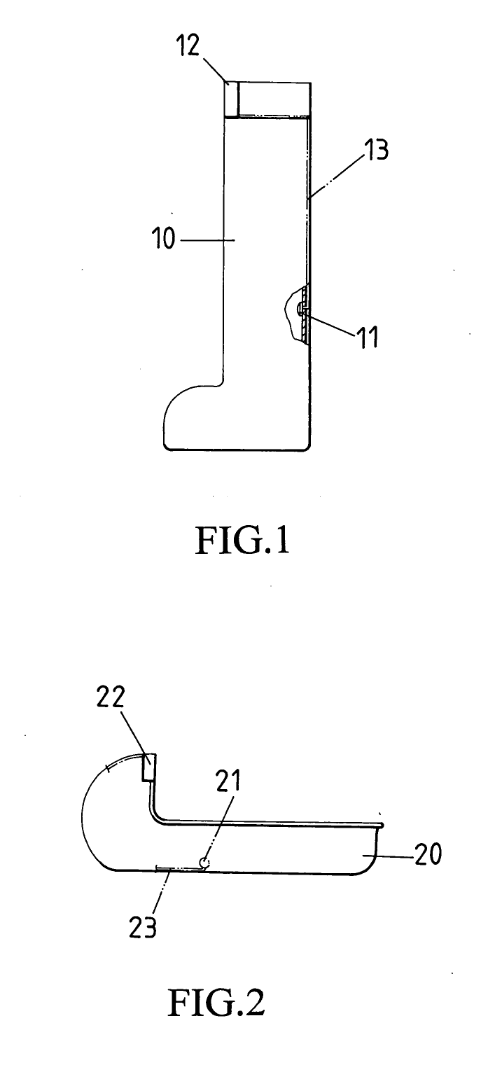

Referring to FIG. 1, it shows a schematic view of a standing urinal for a male, wherein a proper position in an interior of the urinal 10 is installed with a sensor 11, a top end of the urinal 10 is installed with a temperature displayer 12, and an electric wire 13 is connected between the sensor 11 and the temperature displayer 12 to conduct with a power supply. When a male urinates in the urinal 10, and the urine is in touch with the sensor 11, the body temperature can be transmitted to the temperature displayer 12 for displaying, so as to facilitate an acquisition of the physiological condition at that time.

Referring to FIG. 2, it shows a schematic view of a squatting urinal for both a male and a female. A proper position at bottom of the urinal 20 is installed with a sensor 21, a top part of the urinal 20 is installed with a temperature displayer 22, and an electric wire 23 is connected between the sensor 21 and the temperature displayer 22 to conduct with a power supply. When a user urinates in the urinal 20, and the urine is in touch with the sensor 21, the body temperature can be transmitted to the temperature displayer 22 for displaying, so as to facilitate an acquisition of the physiological condition of oneself.

Accordingly, the habit that animals will excrete periodically and the principle that the body temperature is equal to the urine temperature are used by the present invention, to install the sensor and the temperature displayer on the urinal which is commonly used and should be used in a daily life, such that the body temperature can be measured conveniently at will, thereby immediately acquiring the body temperature and the physiological condition at that time.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. A urinal which detects body temperature being a standing or a squatting urinal, with a proper position in an interior of which being installed with a sensor, and with a top part of which being installed with a temperature displayer, such that when urine excreted by a human is in touch with the sensor, the body temperature is immediately displayed on the temperature displayer.

2. The urinal which detects body temperature according to claim 1, wherein an electric wire which conducts with a power supply is connected between the sensor and the temperature displayer.
